(19) United States
(12) Patent Application Publication
(10) Pub. No.: US 2015/0297906 A1
(43) Pub. Date: Oct. 22, 2015

(54) AUTOMATED EXTERNAL DEFIBRILLATOR CABINET DEVICE

(71) Applicant: HD1PY, Inc, Tampa, FL (US)

(72) Inventor: Robert GUICHET, Canohes (FR)

(73) Assignee: HD1PY, Inc, Tampa, FL (US)

(21) Appl. No.: 14/254,436

(22) Filed: Apr. 16, 2014

(51) Int. Cl. A61N 1/39

(52) U.S. Cl. CPC A61N 1/3968

(57) ABSTRACT

The automated external defibrillator cabinet device includes a defibrillator and a housing in which the defibrillator is disposed. There is a device appropriate for signaling the removal of the defibrillator from the housing. There is a link connecting the housing to the defibrillator with a device controlling the operation of the device for signaling in case the link is broken.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The invention relates to a cabinet device for automated external defibrillator (hereinafter AED, a generic term that includes “Fully Automatic Defibrillator” and “Semi-Automatic Defibrillators”), in the form of a housing comprising at least one removable wall to access the AED disposed within and visual and/or audible means of signaling that the AED has been removed from the housing.

The present invention relates to the field of AEDs intended to be installed in any public or private location, in a manner to be easily accessible when needed. It should be noted that AEDs provide an electric shock only if the heart rate analysis so requires (confirmed ventricular fibrillation).

2. Description of Related Art Including Information Disclosed Under 37 CFR 1.97 and 37 CFR 1.98

CRA (Cardiorespiratory Arrest), more commonly called sudden cardiac death or even heart attack, is the second leading cause of mortality in developed countries.

It is a known fact that, during CRA, before the heart completely stops, it generally goes through a phase of arrhythmia known as ventricular fibrillation.

During ventricular fibrillation, the heart no longer acts as a pump and the blood flow is interrupted.

Ten to twelve minutes is the maximum time during which it is possible to restore the heartbeat without severe neurological sequelae. Beyond that time, the non-oxygenated brain may be severely damaged.

The purpose of the AED is to analyze the heart rhythm using two electrodes placed on the chest of the victim, before sending an electric shock enabling the heart to recover an effective beat.

The extreme urgency issue can be addressed only by a widespread, visible deployment of AEDs, both inside and outside of public and private buildings.

Rapid AED use improves the survival rate. The time between the onset of cardiac arrest and use of the AED is a key success factor when trying to restart the heart normally.

Thus more and more public and private locations have been equipped with AEDs. Public availability of AEDs is commonly known as: PAD (Public Access Defibrillation).

However, such public availability of these devices raises fundamental issues, namely:
Keeping the AED in storage conditions compliant with the manufacturer's recommendations;
Facilitating visual inspection that does not always involve opening the cabinet;
Using shapes, colors, and logos to signal the presence of the AED;
Continuously monitoring the proper functioning of the device; and
Ensuring with certainty that it is present in the location where it was installed.

An AED is usually disposed within a cabinet at least one of whose walls can be opened to remove the AED. Thus, the cabinet's primary function is to protect the AED against any damage due to external factors, such as water, dust, or even excessive temperature changes. Its purpose is also to ensure that the AED is present in the location where it was installed.

In particular, as indicated above, the cabinet comprises a wall designed to allow access to the AED. This wall is intended to be retractable or removable and is present in the form of a door or lid through which it is possible to grasp the AED disposed in the cabinet.

It is already known that access to the AED can be detected through this retractable and/or removable wall of the cabinet. In other words, the opening of said wall is detected by appropriate detection means such as sensors, switches, etc., to control the operation of visual and/or audible means of signaling that the AED has been accessed. In some cases, these cabinets are also equipped with appropriate means of transmission to transmit information to an intervention unit or maintenance service. However, it should be noted that opening the cabinet does not necessarily mean that the AED has been removed.

It is also already known that a sensor can be disposed within the cabinet, for example, associated with a shelf on which the AED is disposed, and said sensor is capable of controlling the operation of a visual or audible signal the moment the AED is removed from that shelf. Again, this visual or audible signal will be emitted as soon as a change in the state of an element of that AED housing is detected and not when it is found that the AED has been removed from the cabinet. Furthermore, this sensor may malfunction over time. In fact, not being used on a regular basis, it may function randomly after a period of non-activation due, for example, to partial corrosion, the presence of dust, etc.

### SUMMARY OF THE INVENTION

The purpose of the present invention is an AED cabinet capable of transmitting reliable information, whether visual and/or audible, that the AED has been removed from said cabinet.

Another purpose of the invention is to equip the cabinet with means capable of triggering said visual and/or audible signal by acting on the AED and not on the cabinet itself.

Especially, the purpose of the present invention is to possess the means for activation of said visual and/or audible signaling means without risk of malfunction over time.

Thus the invention relates to an automated external defibrillator cabinet device comprising a defibrillator and a housing in which the defibrillator is disposed; signaling means appropriate for signaling the removal of the defibrillator from the housing comprise a link connecting the housing to the defibrillator, and control means control the operation of the signaling means in case the link is broken. figures.

### BRIEF DESCRIPTION OF THE DRAWINGS

The invention will be better understood upon reading the following description of some embodiments illustrated in the accompanying drawings.

FIG. 1 is a schematic perspective view of an automated external defibrillator cabinet device (hereinafter AED) according to the invention.

FIG. 2 is a similar schematic view with the cabinet open.

FIG. 3 is a partial schematic view of the device according to the invention, according to a first embodiment.

FIG. 4 is a partial schematic view similar to FIG. 3 corresponding to a second embodiment.

FIG. 5 is a similar schematic view corresponding to a third embodiment.

FIG. 6 is a similar schematic view corresponding to a fourth embodiment.

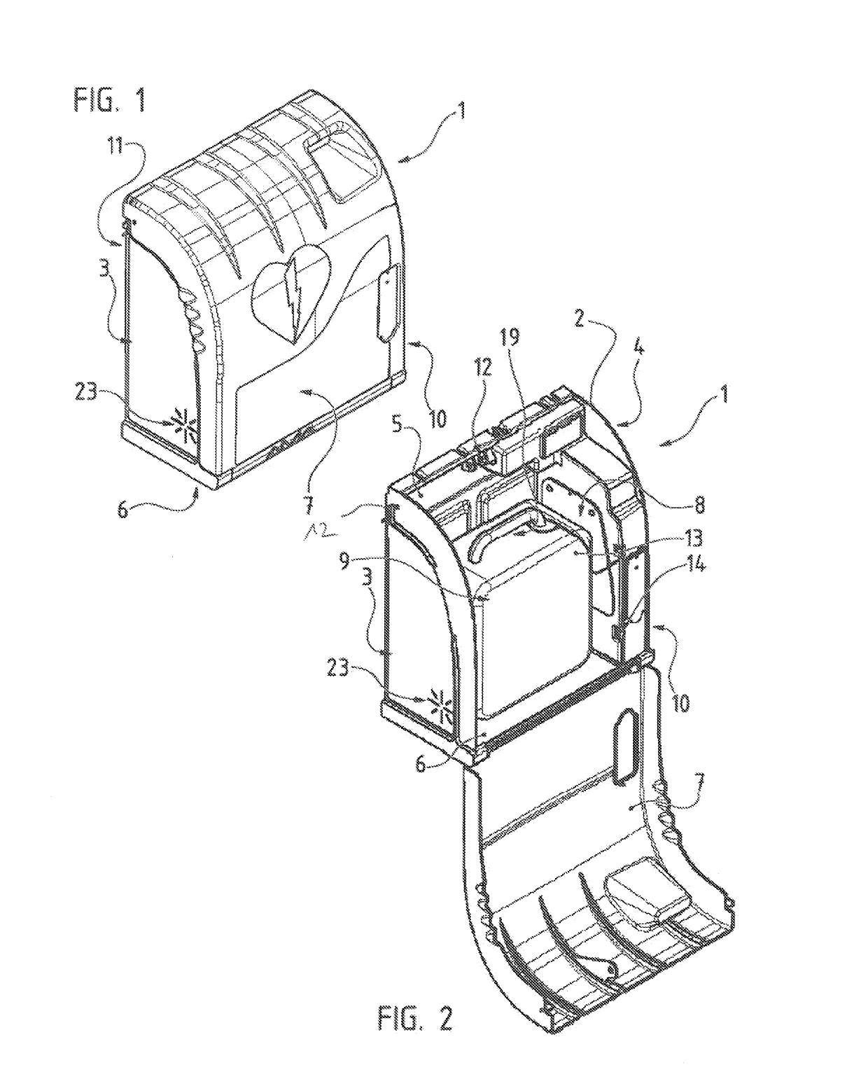

### DETAILED DESCRIPTION OF THE DRAWINGS

As shown in the figures in the accompanying drawings, the present invention relates to an AED cabinet device 1.

This cabinet device 1 comprises a housing 2 delimited by a number of peripheral walls 3, 4, 5, 6, 7 defining a container 8, within which the AED 9 can be positioned. In particular, at least one of these peripheral walls 3, 4, 5, 6, 7 is designed to be removable or movable so as to allow access to the inside of the housing 2 and thus to the AED 9.

Advantageously, this peripheral wall 7 is present in the form of a hood on the front part 10 of the housing 2, opposite the peripheral wall 5 located on the rear part 11 of said housing 2. As an example, said housing 2 can be fastened to a structure through this rear peripheral wall 5.

Still more particularly, the lower part of this removable wall 7 forming a hood can be pivotally mounted on the lower peripheral wall 6 of the housing 2, while the means of closure 12 can maintain it closed on the housing 2. Thus, such closure means 12 can be designed to allow intentional opening of this peripheral wall 7, when the user wishes to access the AED 9.

Preferably, at least the front wall 7 is composed of a transparent material so as to make the AED 9 visible when disposed within the housing 2.

This AED 9 can be contained in a protective jacket 13, shown in the form of a briefcase in the drawings, however this jacket 13 may also take the form of a pouch made of flexible material. It should be noted that the AED 9 is not necessarily contained in such a protective jacket 13 and can be positioned without one in the housing 2 of the cabinet device 1.

According to the invention, this cabinet device 1 also comprises visual and/or audible signaling means 14 designed to transmit information that the AED 9 has been accessed and, preferably, removed from the housing 2.

In particular, these signaling means 14 comprise at least one link 15 in the form of a cord, cable, strap, wire, or the like, connecting the AED 9 or the protective jacket 13 containing this AED 9 to the housing 2, such that the AED 9 can be removed from said housing 2 only by breaking this link 15.

Also according to the invention, this link 15 mates via first fastening means 16 with the housing 2, and via second fastening means 17 with the AED 9.

These second fastening means 17 are shown in the figures of the accompanying drawings in the form of a handle 18 on the briefcase corresponding to the protective jacket 13 containing the AED 9.

Finally, the second fastening means 17 are defined in the form of an opening 19 in the closed contour 20 worked in the protective jacket 13 or in the AED 9 directly, and the link 15 is able to pass through said opening 19.

Very substantially, such an opening 19 may be present in the form of a loop, a handle, a hole, either in the protective jacket 13 or in the AED 9 itself.

Complementarily, the signaling means 14 comprise control means 21 for operating at least one visual signaling device 22 (for example, a light) and/or at least one audible signaling device 23 (for example, a speaker). The one and/or the other of these visual 22 and/or audible 23 signaling devices may be implanted on the cabinet device 1, since it is also possible to consider installing them remotely using appropriate wired or non-wired means of transmission.

To return to the operation control means 21, these consist of means of very concretely detecting the opening or detachment of the link 15.

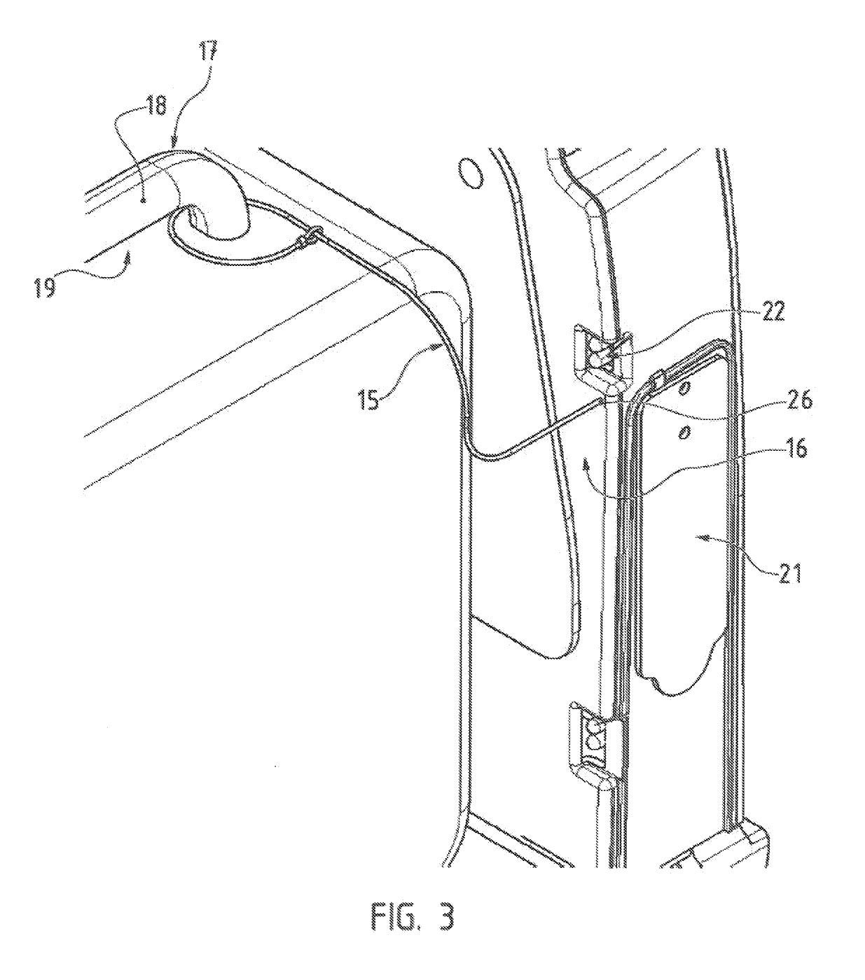

Thus, as illustrated in the embodiment in FIG. 3, the link 15 is in the form of a cord 24 which, via a loop 25 mating with the second fastening means 17, is connected to the AED 9 or more precisely, in the illustrated embodiment, to the handle 18 of the protective jacket 13 containing the AED 9, since that cord 24 also comprises first fastening means 16 in the form of a plug 26 engaging in a complementary plug in the housing 2 and associated with the control means 21. The loop 25 and the opening 19 mate in the manner of two links in a chain.

Thus, in case the plug 26 is removed from the cord 24, the operation control means 21 detect the removal of the AED 9 and control the operation of the visual 22 and/or audible 23 signaling device(s).

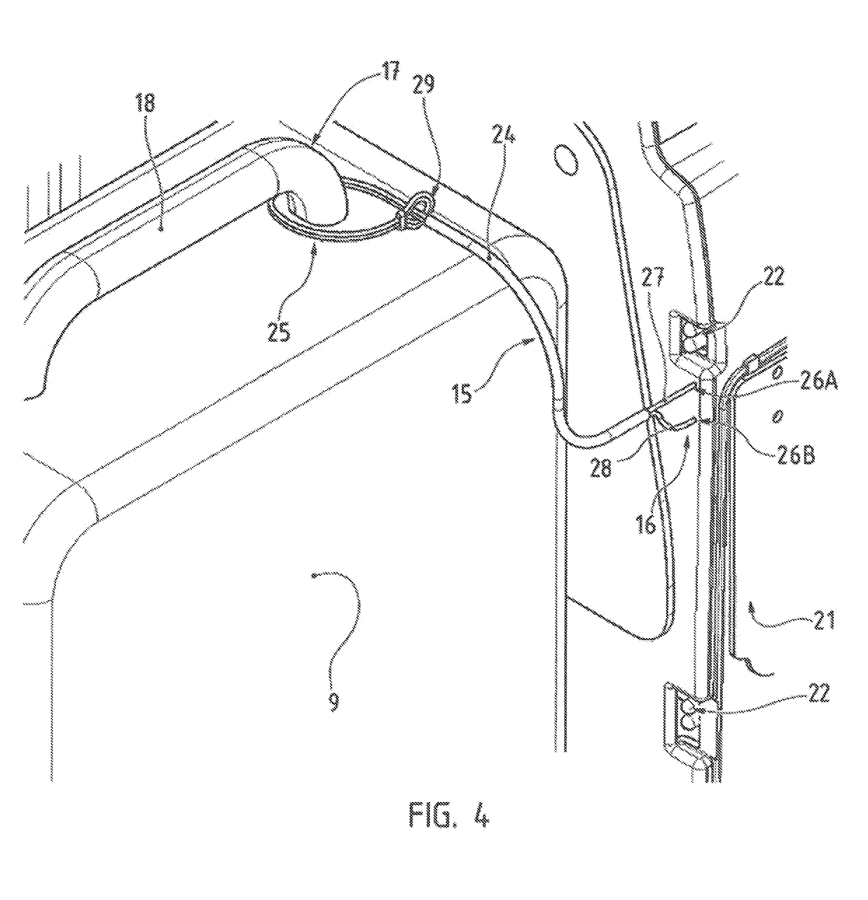

In the embodiment illustrated in FIG. 4, the link 15 is again defined by a cord 24 mating with the AED 9 through a loop 25 and by means of a plug 26 [mating] with the control means 21 associated with the housing 2.

Advantageously, the cord 24 is defined, in this embodiment, by two conductor wires 27, 28, which together describe a loop circuit 29, the plug 26 having substantially the form of a double plug one of which 26A contributes to connecting the first conductor wire 27 and the other 26B to connecting the second conductor wire 28 to a complementary plug similar to these control means 21, such that tearing or cutting the cord 24 corresponding to the link 15 and releasing the plug 26 is interpreted as a removal of the AED 9 from the housing 2 by opening the loop circuit 29.

In sum, the control means 21 consist of an opening sensor of a loop circuit 29 for controlling the operation of visual 22 and/or audible 23 signaling devices.

In both embodiments described above, the removal of the AED 9 from the housing 2 leads simultaneously to the removal of the link 15 from the cabinet device 1.

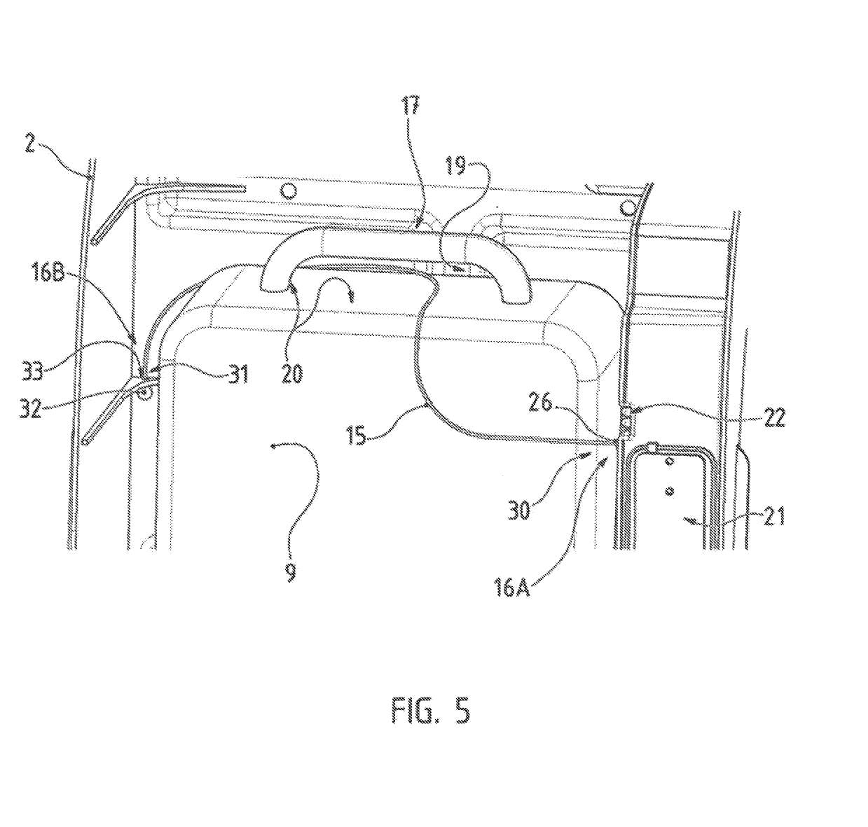

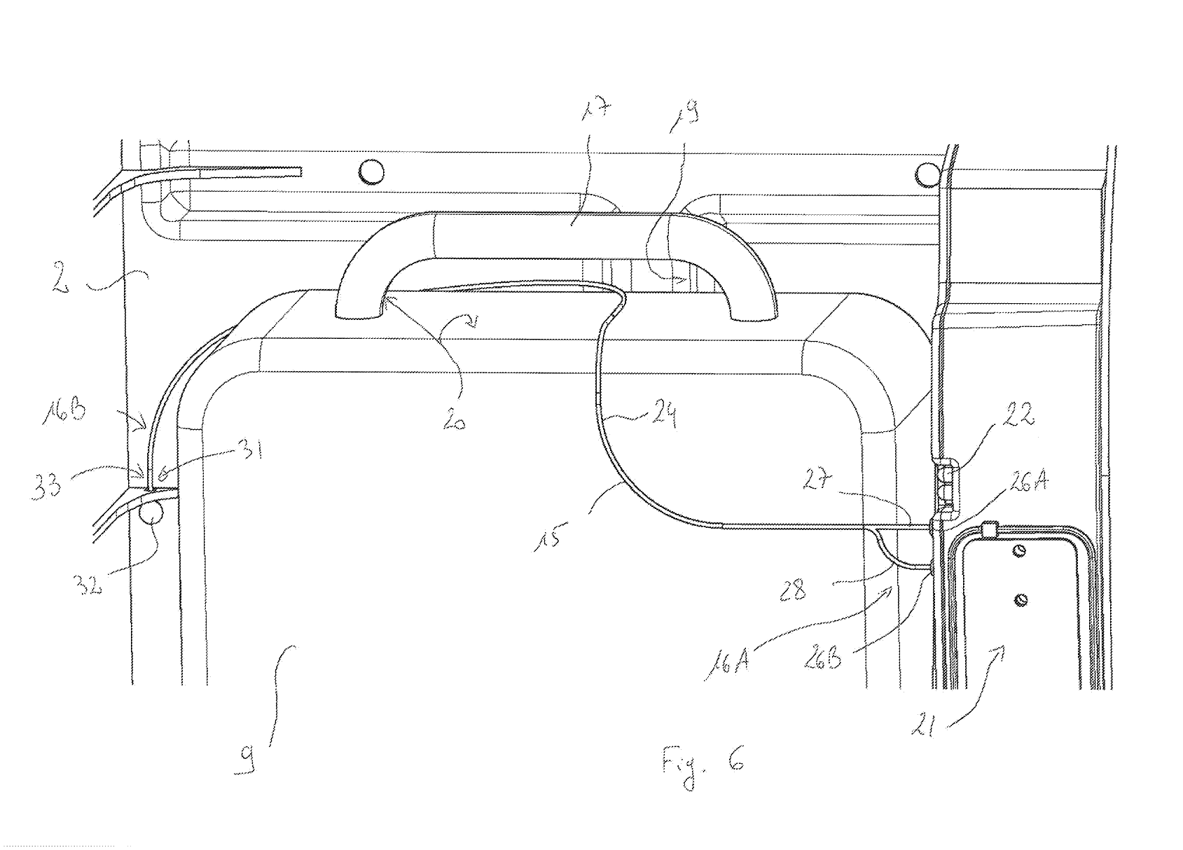

To avoid this consequence, the embodiment illustrated in FIG. 5 was designed, such that, at each of its two ends 30, 31, the link 15 mates with the housing 2 via the first fastening means 16A, 16B, and via the second fastening means 17 (defined by the opening 19 in the closed contour 20 in the AED 9 or its protective jacket 13, with the link 15 passing through said opening 19) with the AED 9.

Furthermore, at only a first end 30 of this link 15, the fastening means 16A are defined as removable in order to detach from the housing 2 in case the AED 9 is removed.

In particular, at this first end 30, the link 15 can mate, by means of a plug 26, with a complementary plug that the control means 21 comprise, similarly to the first embodiment described above and illustrated in FIG. 3.

At the second end 31 of the link 15, the fastening means 16B are preferably designed to be non-removable. In particular, as shown in FIG. 5, at this end 31, the link 15 may terminate in a tip in the form of a ball 32 defining a stop against a grommet 33 fitted to the housing 2. Thus, the first end 30 (with the plug 26) is threaded through this grommet 33 until it brings the ball 32 to stop against the grommet 33. The first end 30 of the link 15 is then passed through the opening 19 in the closed contour 20 defining the second fastening means 17 in the AED 9 or the protective jacket 13 containing it. Then the plug 26 at the first end 30 is engaged in the complementary plug corresponding to the control means 21.

Thus, to use the AED 9, it is removed from the housing 2 after it is opened. When it is removed, the plug 26 (at the first end 30 of the link 15) is disconnected, resulting in the operation of the visual 22 and/or audible 23 signaling means under the influence of the control means 21.

FIG. 6 shows another embodiment wherein, as in FIG. 5, at each of its two ends 30, 31, the link 15 mates via the first fastening means 16A, 16B with the housing 2, and via the second fastening means 17 (as defined by the opening 19 in the closed contour 20 in the AED 9 or its protective jacket 13, with the link 15 passing through said opening 19) with the AED 9. In addition, as in FIG. 4, the link 15 is a cord 24 that comprises two conductor wires 27, 28, which together describe a loop circuit 29, the plug 26 having the form of a double plug one of which 26A contributes to connecting the first conductor wire 27 and the other 26B to connecting the second conductor wire 28 to a complementary plug similar to these control means 21, such that tearing or cutting the cord 24 and releasing the plug 26 is interpreted as a removal of the AED 9 from the housing 2 by opening the loop circuit 29.

The advantages of the present invention are that the visual 22 and/or audible 23 signaling device(s) are activated only through the actual removal of the AED 9 from the cabinet device 1. In particular, for maintenance purposes, for example, the AED 9 can be manipulated in the housing 2 to make all the necessary checks, without this manipulation or even the opening of the cabinet device 1 generating the operation of the visual 22 and/or audible 23 signaling means.

1. Automated external defibrillator cabinet device comprising:
a defibrillator;
a housing, said defibrillator being disposed in said housing;
signaling means for removal of the defibrillator from the housing;
a link connecting the housing to the defibrillator; and
control means for operation of the signaling means in case the link is broken.

2. Cabinet device according to claim 1, wherein the link mates via first fastening means with the housing, and via second fastening means with the defibrillator.

3. Cabinet device according to claim 2, wherein the link comprises two ends, one mating with the first fastening means and the other with the second fastening means.

4. Cabinet device according to claim 2, wherein the link comprises two ends mating with two first fastening means and another part of the link mating with the second fastening means.

5. Cabinet device according to claim 2, wherein the second fastening means are comprised of an opening in a closed contour worked in the defibrillator, and the link passes through said opening.

6. Cabinet device according to claim 3, wherein the second fastening means are comprised of an opening in a closed contour worked in the defibrillator, and the link passes through said opening, and wherein the link mates through a loop engaged with the opening, in the manner of links of a chain.

7. Cabinet device according to claim 4, wherein the second fastening means are comprised of an opening in a closed contour worked in the defibrillator, and the link passes through said opening, and wherein the link passes through the opening.

8. Cabinet device according to claim 2, wherein the first fastening means are removable and are comprised of a plug engaging in a complementary plug worked in the housing and associated with control means.

9. Cabinet device (1) according to claim 2, wherein the link is defined by a cord comprised of two conductor wires, forming a loop circuit, one end of the link mated with the first fastening means being comprised of a double plug, one plug contributing to connecting a first conductor wire and the other plug to connecting a second conductor wire, the double plug being connected to a complementary plug associated with control means.

10. Cabinet device according to claim 4, wherein only a first end removably mates with the corresponding first fastening means.
